(19) United States
(12) Patent Application Publication
(10) Pub. No.: US 2026/0069651 A1
(43) Pub. Date: Mar. 12, 2026

(54) Application of Sardine Peptide Composition in Drug for Treating Allergic Rhinitis

(71) Applicant: HAIKOU QIAOSHUI UNITED TECHNOLOGY CO. LTD., Haikou (CN)

(72) Inventors: Jiaxi WANG, Haikou (CN); Jinna CAO, Haikou (CN); Rubin PENG, Haikou (CN); Ji ZHOU, Haikou (CN); Hongbin ZHANG, Haikou (CN); Huicheng LI, Haikou (CN)

(73) Assignee: HAIKOU QIAOSHUI UNITED TECHNOLOGY CO. LTD., Haikou (CN)

(21) Appl. No.: 19/387,560

(22) Filed: Nov. 12, 2025

Related U.S. Application Data
(63) Continuation of application No. PCT/CN2024/091829, filed on May 9, 2024.

(30) Foreign Application Priority Data
May 16, 2023 (CN) 202310548514.X

(51) Int. Cl. A61K 38/05; A61K 38/06; A61K 38/07; A61K 38/08; A61P 37/08

(52) U.S. Cl. CPC A61K 38/05; A61K 38/06; A61K 38/07; A61K 38/08; A61P 37/08

(57) ABSTRACT

The present invention provides an application of a sardine peptide composition in a drug for treating allergic rhinitis. The sardine peptide composition is a mixture of dipeptide to pentapeptide of sardine. The biological activities of various peptide fragments are synergistic. The sardine medicine composition has the effects of inhibiting nasal cavity inflammation attack and preventing nasal cavity capillary obstruction, and can also improve rhinocnesmus, sneezing, rhinorrhea and nasal obstruction caused by allergic rhinitis, as well as various symptoms accompanied with itching of palate, throat, ears and eyes, red and swollen eyes and water secretions. Meanwhile, the sardine peptide composition has no irritation to patients with nasal mucous membrane sensitivity, systemic side effects such as local side effects and drowsiness are not observed, and the sardine peptide composition has excellent effectiveness and safety.

### CROSS-REFERENCE TO RELATED APPLICATION

This application is a continuation of the international application PCT/CN2024/091829 filed on May 9, 2024, which claims the priority benefit to China application CN202310548514.X filed on May 16, 2023. The contents of the above identified applications are herein incorporated by reference in their entirety and made a part of this specification.

### TECHNICAL FIELD

The present invention belongs to the technical field of biological medicine and specifically relates to an application of sardine peptide composition in drug for treating allergic rhinitis.

### BACKGROUND

Allergic rhinitis, also known as AR, is an inflammatory nasal cavity disease mediated by immunoglobulin E (IgE), which affects about 10% of the population, and is caused by the exposure of sensitive individuals to allergens. Mast cells, eosinophils, CD4+ T lymphocytes and B lymphocytes are the main inflammatory cells involved. Mast cell degranulation releases various transmitters, which leads to increased microvascular permeability, nasal mucous membrane edema, water secretions, and release of various cytokines, and is an important mechanism of tissue damage in allergic rhinitis.

Angiotensin II (Ang II) is an important constituent part of a renin-angiotensin system (RAS). Most of the effects of Ang II are mediated by AT1. The combination of the mast cells and Ang II can cause vasoconstriction, increased blood pressure, and pro-inflammatory effects on white blood cells, endothelial cells, and vascular smooth muscle cells. Angiotensin converting enzyme (ACE) is a zinc metalloprotease, mainly present in pulmonary vascular endothelial cells, but also in most tissues such as uterus, placenta, heart, blood vessels, kidney, brain, and adrenal cortex. ACE converts AngI to AngII (vasoconstrictor) and degrades bradykinin (vasodilator), regulating blood pressure and cardiovascular homeostasis in vivo. AngII can increase the vascular permeability by inducing mast cell degranulation.

Mast cell (MC) degranulation is an important link in allergic reactions. And MC degranulation is caused by the regulation of calcium influx and the activation of calcium signaling pathways. The current drug for allergic rhinitis, disodium cromoglycate (DSCG), restricts a chloride ion channel with a function of regulating cell volume, prevents extracellular calcium ions from flowing into the mast cell cytoplasm, and thus prevents mast cell degranulation. DSCG can limit the nasal cavity response to allergens, making it one of the best options for preventing the onset of symptoms. Studies have found that VY, i.e., Val-Tyr, also has a function of inhibiting calcium ion channels, and can inhibit the influx of extracellular calcium ions.

Allergic rhinitis is a recurrent disease that requires continuous use of drugs. At present, how to effectively treat allergic rhinitis is still a difficult problem in the medical field. Existing steroid drugs for nasal sprays have nasal irritation, dryness, burning sensation in the nose, epistaxis and the like. Antihistamines can suppress allergic reactions, but they also have disadvantages such as dry nasal mucous membrane and drowsiness. Therefore, it is of an important practical significance to provide a drug that can effectively treat allergic rhinitis.

### SUMMARY

The present invention proposes an application of a sardine peptide composition in a drug for treating allergic rhinitis. Studies have confirmed that the sardine peptide composition has excellent effectiveness and safety as a therapeutic agent for allergic rhinitis, and can significantly improve symptoms associated with rhinitis, in particular allergic rhinitis.

A sardine peptide is a mixture of oligopeptides obtained by processing sardine muscles, with a molecular weight of 200-900 daltons. At present, the sardine peptide has been added to food and health food to maintain normal blood pressure levels. The oligopeptides contained in the sardine peptide have high biological activity, and are divided into anti-allergic active short peptides, ACE-inhibiting active short peptides and L-type calcium ion channel-inhibiting active peptides according to their functions. Therefore, the inventors conducted further research on the functions of each active peptide and found that anti-inflammatory active peptides can prevent and treat inflammations by reducing the release of pro-inflammatory cytokines, inhibiting or intervening in TNF-α-induced inflammatory pathways, as well as NF-κB and MAPK signaling pathways. The ACE-inhibiting short peptides inhibit the activity of ACE and the production of Ang II, thereby inhibiting inflammations. The L-type calcium ion channel-inhibiting active peptides can inhibit the activity of L-type calcium channels in cells, thereby inhibiting the influx of calcium ions and preventing mast cell degranulation. The physiological mechanisms of these bioactive polypeptides support the role of sardine peptides in the treatment of allergic rhinitis or symptom improvement. Based on this, the inventors further found that the absorption efficiency of mixed polypeptides is higher than that of single polypeptides, the biological activities between polypeptides have a synergistic effect, and the biological activities of mixed polypeptides are higher than those of single polypeptides.

In order to solve the above technical problem, the present invention provides an application of a sardine peptide composition in a drug for treating allergic rhinitis. Further, the sardine peptide composition is prepared from sardine by means of a microbial fermentation method, an acid method, an alkaline method, an electrical method, an artificial grafting method, a gene expression method, and an enzymolysis method.

Further, the sardine peptide composition is a mixture of dipeptide to pentapeptide of sardine.

Further, in the sardine peptide composition, the sardine dipeptide comprises Ala-Leu, Lys-Trp, Ala-Trp, Met-Leu, Arg-Tyr, Met-Phe, Gly-Trp, Met-Tyr, Gly-Tyr, Phe-Leu, Ile-Phe, Thr-Tyr, Ile-Trp, Tyr-Leu, Ile-Tyr, Val-Phe, Leu-Trp, Val-Trp, Leu-Tyr and Val-Tyr; the sardine tripeptide comprises Ala-Asn-Pro, Ala-Lys-Lys Arg-Phe-His, Arg-Val-Tyr, Gly-Arg-Pro, Ile-Val-Tyr, Trp-Ile-Thr, Tyr-Val-Pro and Val-Val-Phe; the sardine tetrapeptide is Gly-Trp-Ala-Pro; and the sardine pentapeptide is Asn-Val-Pro-Ile-Tyr.

The present invention further provides a composition for preventing and treating allergic rhinitis, wherein containing a sardine peptide composition and pharmaceutically acceptable auxiliary materials

Further, the sardine dipeptide comprises Ala-Leu, Lys-Trp, Ala-Trp, Met-Leu, Arg-Tyr, Met-Phe, Gly-Trp, Met-Tyr, Gly-Tyr, Phe-Leu, Ile-Phe, Thr-Tyr, Ile-Trp, Tyr-Leu, Ile-Tyr, Val-Phe, Leu-Trp, Val-Trp, Leu-Tyr and Val-Tyr; the sardine tripeptide comprises Ala-Asn-Pro, Ala-Lys-Lys Arg-Phe-His, Arg-Val-Tyr, Gly-Arg-Pro, Ile-Val-Tyr, Trp-Ile-Thr, Tyr-Val-Pro and Val-Val-Phe; the sardine tetrapeptide is Gly-Trp-Ala-Pro; and the sardine pentapeptide is Asn-Val-Pro-Ile-Tyr.

Further, a mass concentration of the sardine peptide composition in the composition is 0.01%-50%, preferably 0.15%-10% (w/w).

The number and duration of use of the composition depend on different symptoms, ages, dosage forms, etc., but the standard number is 2-3 times a day, and the duration of use is 7-14 days.

Further, administration routes of the composition can be used according to patients and symptoms, including: a mucous membrane administration preparation, a gastrointestinal tract administration preparation, a respiratory tract administration preparation, a skin administration preparation, a cavity administration preparation, and an injection administration preparation. Specifically, the composition may be nasal drops, gel, ointment, cream, lotion, spray, liquid preparation, nasal spray, patch, aerosol, jelly, cataplasm, patch, plaster, suspension, emulsion, injection, tablets, pills, capsules, granules, powder, etc. The composition may also be used as a liquid preparation by selecting an appropriate solvent. Any preparation may be prepared by a known method. Further, the mucous membrane administration preparation is preferably nasal preparations or sublingual tablets; and the nasal preparations include ointment, gel, cream, lotion, liquid preparation, powder, oil spray or spray; and preferably, oil spray. The nasal drops of the present invention may be liquid preparations, powder preparations or other dry products, and may contain carriers or excipients, surfactants, suspension aids, osmotic pressure regulators, etc. Sodium chloride, glycerin, sodium bisulfite, benzalkonium chloride, fructose, citric acid, sodium citrate, crystalline sodium dihydrogen phosphate, sodium hydroxide, sorbitol liquid, niacinamide, concentrated glycerin, propylene glycol, benzyl alcohol, boric acid, borax, polyethylene glycol 4000, sodium hydroxide, phosphoric acid, sodium dihydrogen phosphate, and potassium dihydrogen phosphate may be used. Crystalline cellulose, sodium carboxymethyl cellulose and hydroxypropyl cellulose may be used as suspension aids. The liquid preparation is prepared by dissolving the sardine peptide composition as an active ingredient in ethanol, glycerin, propylene glycol, polyethylene glycol, water or fatty oil as a matrix. A liquid preparation containing an aqueous solution prepared by dissolving the sardine peptide composition in normal saline is preferred. In addition to normal saline, the aqueous solution may also be mixed with a small amount of organic solvent such as ethanol, glycerin, propylene glycol, polyethylene glycol, or dimethyl sulfoxide.

The liquid preparation, in addition to the aqueous solution preparation mentioned above, may also form an aqueous or oil solution, a suspension, an emulsion, etc. In addition, the composition may also be provided as a dry drug composition redissolved in water or an appropriate medium before use.

The spray is formed by preparing the sardine peptide composition into an aqueous solution, an oil solution, an emulsion or a suspension.

During the preparation of the therapeutic agent for rhinitis of the present invention, various matrices, moisturizers, alcohols, chelating agents, pH adjusters, preservatives, thickeners, colorants, fragrances, fillers, excipients, disintegrants, extenders, adhesives, smears, solubilizers, suspension aids, buffers, stabilizers, bacteriostatic agents, surfactants, antioxidants, emulsifiers, co-solvents, etc., may be combined and matched in any way. In addition to the sardine peptide as an active ingredient, various agents, such as vitamins and bactericidal disinfectants can also be appropriately matched as needed.

Further, the allergic rhinitis (AR) comprises seasonal allergic rhinitis and perennial allergic rhinitis.

### The Beneficial Effects of the Present Invention are as Follows

1. The sardine peptide composition of the present invention contains a variety of short peptides with high biological activity, which are divided into anti-allergic active short peptides, ACE-inhibiting active short peptides and L-type calcium ion channel-inhibiting active peptides according to their functions. The anti-inflammatory active peptides can prevent and treat inflammations by reducing the release of pro-inflammatory cytokines, inhibiting or intervening in TNF-α-induced inflammatory pathways, as well as NF-κB and MAPK signaling pathways. The ACE-inhibiting short peptides inhibit the activity of ACE and the production of Ang II, thereby inhibiting inflammations. The L-type calcium ion channel-inhibiting active peptides can inhibit the activity of L-type calcium channels in cells, thereby inhibiting the influx of calcium ions and preventing mast cell degranulation. The different short peptides in the sardine peptide composition cooperate with each other and exert their respective effects, so that the composition has a significant effect in the treatment and improvement of allergic rhinitis-related symptoms.

2. The sardine peptide composition of the present invention has a good effect in the treatment of allergic rhinitis or symptom improvement, and can be prepared into a variety of dosage forms for administration.

3. The nasal preparation prepared from the sardine peptide composition of the present invention improves the nasal cavity capillary obstruction, and can also improve rhinocnesmus, sneezing, rhinorrhea and nasal obstruction caused by allergic rhinitis, as well as itching of palate, throat, ears and eyes, red and swollen eyes and water secretions. Further, the nasal preparation has no irritation to patients with nasal mucous membrane sensitivity, and systemic side effects such as local side effects and drowsiness are not observed.

### BRIEF DESCRIPTION OF THE DRAWINGS

The invention is further described below with reference to the accompanying drawings and embodiments.

FIG. 1 shows effects of drugs in Test Example 2 on the cilia movement of the toad palate;

FIG. 2 shows a state diagram of the cilia movement at the initial time of administration in Test Example 2; and

FIG. 3 shows a state diagram of the cilia movement at 18 h after administration in Test Example 2.

In drawings, A represents normal saline; B represents refined sesame oil; C represents sardine peptide nasal spray (oil solution) in Embodiment 4; and D represents a sardine peptide nasal spray (oil solution)+berberine sesame oil suspension in Embodiment 4.

### DETAILED DESCRIPTION OF THE INVENTION

In the following embodiments, unless otherwise specified, all the experimental instruments and raw materials mentioned are commercially available products. Raw materials were shown in Table 1.

TABLE 1

| Material name | Grade | Source |
|---|---|---|
| Sardine peptide | Food grade | Senmi Ekisu, Japan |
| composition | | |
| (Valtyron) | | |
| Cyclodextrin | Food grade | Sichuan Huayuan Shengtai |
| | | Biological Technology Co., LTD |
| Dipotassium | Food grade | Sichuan Kangbai Rui Biological |
| hydrogen | | Technology Co., LTD |
| phosphate | | |
| Potassium | Food grade | Sichuan Kangbai Rui Biological |
| dihydrogen | | Technology Co., LTD |
| phosphate | | |
| Sodium chloride | Reagent grade | Zhongke Ruitai (Beijing) |
| | | Biotechnology Co., LTD |
| benzalkonium | Pharmaceutical | Xi 'an Tianzheng Pharmaceutical |
| chloride | grade | Excipients Co., LTD |
| Vitamin E | Food grade | Zhejiang Xinhua Cheng Co., LTD |
| Refined | Food grade | Croda Inc. |
| sesame oil | | |
| Soybean | Food grade | Shenyang Tianfeng Biological |
| lecithin | | Pharmaceutical Co., LTD |
| Soybean | Pharmaceutically | Shanxi Jinyang Pharmaceutical |
| oil | oral grade | Excipients Co., LTD |

It was determined that Valtyron, a sardine peptide composition of Senmi Ekisu Co. Ltd., was prepared by enzymatic hydrolysis. The sardine peptide composition is a mixture of dipeptide to pentapeptide of sardine. In the sardine peptide composition, the sardine dipeptide comprises Ala-Leu, Lys-Trp, Ala-Trp, Met-Leu, Arg-Tyr, Met-Phe, Gly-Trp, Met-Tyr, Gly-Tyr, Phe-Leu, Ile-Phe, Thr-Tyr, Ile-Trp, Tyr-Leu, Ile-Tyr, Val-Phe, Leu-Trp, Val-Trp, Leu-Tyr and Val-Tyr; the sardine tripeptide comprises Ala-Asn-Pro, Ala-Lys-Lys Arg-Phe-His, Arg-Val-Tyr, Gly-Arg-Pro, Ile-Val-Tyr, Trp-Ile-Thr, Tyr-Val-Pro and Val-Val-Phe; the sardine tetrapeptide is Gly-Trp-Ala-Pro; and the sardine pentapeptide is Asn-Val-Pro-Ile-Tyr.

The experimental instruments were shown in Table 2.

TABLE 2

| Instrument | Model | | Source |
|---|---|---|---|
| High-speed shear | AE500S-H | | Shanghai Onni Instrument Co., LTD |
| emulsifier | | | |
| High-pressure | AH-BASIC | | ATS Industrial Systems Co., Ltd |
| homogenizer | | | |
| Microporous | 0.22 | μm | Beilanbo Biotechnology |
| filter | | | (Hangzhou) Co., LTD |
| membrane | | | |
| Quantitative | 20 | ml | Guangdong Huachuang |
| nasal sprayer | 30 | ml | Pharmaceutical Precision |
| | | | Packaging Co., LTD |

### Embodiment 1: Sardine Peptide Nasal Spray (Aqueous Solution)

The present embodiment provided a sardine peptide nasal spray of an aqueous solution type, including a sardine peptide composition and related auxiliary materials. The content of various materials in the nasal spray was shown in Table 3.

TABLE 3

| Raw material | Content g/ml | Dose(g) | Effects |
|---|---|---|---|
| Sardine peptide | 0.015 | 15 | Main drug |
| composition | | | |
| Cyclodextrin | 0.01 | 10 | Mask the taste and |
| | | | promote absorption |
| Dipotassium | 0.00087 | 0.87 | Buffer |
| hydrogen | | | pH of 5.8 |
| phosphate | | | |
| Potassium | 0.00834 | 8.34 | |
| dihydrogen | | | |
| phosphate | | | |
| Sodium | 0.03 | 30 | Regulate osmotic |
| chloride | | | pressure |
| benzalkonium | 0.0001 | 0.1 | Bacteriostatic |
| chloride | | | agent |

| | |
|---|---|
| Injection water | To 1000 ml |

A preparation method for the sardine peptide nasal spray in the present embodiment included the following steps.

First, various materials were weighed accurately according to the doses shown in Table 3 and dissolved in an appropriate amount of injection water, respectively; and after the materials were completely dissolved, the components were mixed well and added with the injection water to required configuration amounts. The pH value of the measured solution reached 5.5 to 6.5, and the solution was filtered with a 0.22 μm microporous filter membrane to obtain a mixed aqueous solution. The mixed aqueous solution was canned in a 30 ml quantitative spray container, that is, the sardine peptide nasal spray of the aqueous solution type was prepared. The spray volume of this nasal spray was 50 μl per spray.

### Embodiment 2: Sardine Peptide Nasal Spray (Oil Solution)

The present embodiment provides a sardine peptide nasal spray of an oil solution type, including a sardine peptide composition and related auxiliary materials. The content of various materials in the nasal spray was shown in Table 4.

TABLE 4

| Raw material | Content g/ml | Dose(g) | Effects |
|---|---|---|---|
| Sardine peptide composition | 0.015 | 1.5 | Main drug |
| Vitamin E | 0.01 | 1 | Resist oxidation |

| | |
|---|---|
| Refined sesame oil | To 100 ml |

A preparation method for the sardine peptide nasal spray in the present embodiment included the following steps.

The sardine peptide composition and vitamin E were accurately weighed according to the doses shown in Table 4. The vitamin E was first dissolved in sesame oil, and dissolved completely. Then, the sardine peptide composition was suspended in the sesame oil solution, and prepared into the sardine peptide nasal spray of the oil solution type.

The sardine peptide nasal spray was canned into a 20 ml quantitative nasal sprayer with a spray volume of approximately 45 μl per spray under a state of shaking well without precipitate.

### Embodiment 3: Sardine Peptide Nasal Spray (Emulsion)

The present embodiment provides a sardine peptide nasal spray of an emulsion type, including a sardine peptide composition and related auxiliary materials. The content of various materials in the nasal spray was shown in Table 5.

TABLE 5

| | Raw material | Dose | | Effects |
|---|---|---|---|---|
| Aqueous | Sardine peptide | 15 | g | Main drug |
| phase | composition | | | |
| | Purified water | 300 | mL | Aqueous phase |
| Oil phase | Vitamin E | 5 | g | Resist oxidation |
| | Soybean lecithin | 3 | g | Emulsifier |
| | Soybean oil | 700 | mL | Oil phase |

A preparation method for the sardine peptide nasal spray in the present embodiment included the following steps.

First, various materials were accurately weighed according to the doses shown in Table 5, and the sardine peptide composition was dissolved in purified water to form an aqueous phase solution. Vitamin E, soybean lecithin and soybean oil were then stirred and mixed well to obtain an oil phase solution.

Then, the aqueous phase solution was poured into the oil phase solution, and emulsified with a high-speed shear emulsifier at a speed of 1000 rmp to obtain a coarse emulsion. The coarse emulsion was emulsified with a high-pressure homogenizer at a pressure of 70° C. and 1000 bar to prepare a water-in-oil emulsion, i.e., the sardine peptide nasal spray of the emulsion type.

The emulsion was canned in a 30 ml quantitative nasal sprayer, and the spray volume was 45 μl per spray.

### Embodiment 4: Sardine Peptide Nasal Spray (Oil Solution)

The present embodiment provided a sardine peptide nasal spray of an oil solution type, which, similar to the spray of the oil solution type in Embodiment 2, included an sardine peptide composition and related auxiliary materials, with a difference just in that: the content of the sardine peptide composition in the spray was 0.02 g/ml.

### Test Example 1

The sardine peptide nasal sprays of the aqueous solution type, the oil solution type and the emulsion type prepared in Embodiments 1, 2 and 3 were administrated to patients with allergic rhinitis 2 to 3 times a day, and the patient's responses were counted after 7 days of continuous use.

A total of 18 subjects were involved, which were randomly divided into 3 groups, with 6 subjects in each group.

The 6 subjects in the first group used the sardine peptide nasal spray of the aqueous solution type prepared in Embodiment 1, which was sprayed with 1-2 sprays on the nasal cavity in the morning and evening every day, or 1-2 sprays when nasal obstruction was caused by external environmental stimuli. A nozzle should be pointed to the outside of the nasal cavity while spraying.

The 6 subjects in the second group used the sardine peptide nasal spray of the oil solution type prepared in Embodiment 2. A spray bottle was shaken up and down 20 times before use, or no precipitation was observed at the bottom of the bottle. Each subject was sprayed with 1-2 sprays on the nasal cavity in the morning and evening every day, or 1-2 sprays when nasal obstruction was caused by external environmental stimuli. A nozzle should be pointed to the outside of the nasal cavity while spraying.

The 6 subjects in the third group used the sardine peptide nasal spray of the emulsion type prepared in Embodiment 3. Each subject was sprayed with 1-2 sprays on the nasal cavity in the morning and evening every day, or 1-2 sprays when nasal obstruction was caused by external environmental stimuli. A nozzle should be pointed to the outside of the nasal cavity while spraying. After 7 days of continuous use, the effects were shown in Table 6.

TABLE 6

| | | After the drug | | | | |
|---|---|---|---|---|---|---|
| | | was | | | | |
| | | administrated, | | | | |
| | | the onset time | | | | |
| | | of nasal | | | | |
| | Number | obstruction | | | | |
| | of | symptoms | Nasal | | | Experience |
| | patients | was improved | obstruction | Sneezing | Epistaxis | feeling |
| Embodiment | 6 | 5~15 min | All | All | All | Some fishy |
| 1 | | | improved | improved | improved | smell |
| Embodiment | 6 | 10~20 min | All | All | All | Slightly fishy |
| 2 | | | improved | improved | improved | smell, |
| | | | | | | tolerable |
| Embodiment | 6 | 10~20 min | All | All | All | No fishy |
| 3 | | | improved | improved | improved | smell, |
| | | | | | | tolerable |

### Test Example 2: Study on the Toxicity of Sardine Peptide Nasal Spray on Nasal Cilia

In order to detect whether the sardine composition of the present application is toxic to the nasal mucous membrane, the present test example used the in vitro mucous membrane of the toad palate as a model to investigate the cilia toxicity of the sardine peptide nasal spray (oil solution) in Embodiment 4. A specific experimental method was as follows:

### 1. Materials

### 1.1 Test Drugs

A: normal saline;
B: refined sesame oil;
C: sardine peptide nasal spray (oil solution) in Embodiment 4; and
D: sardine peptide nasal spray (oil solution)+berberine sesame oil suspension (berberine concentration of 5 mg/ml) in Embodiment 4.

### 1.2 Instrument

BM1000 optical microscope.

### 1.3 Animals

Bufo gargarizans (about 50 g each) were purchased from the Animal Center of Shenyang Pharmaceutical University.

### 2 Experimental Method

After nasal administration, the nasal mucous membrane may be affected to varying degrees, and the toxicity of the drug to the nasal mucous membrane was one of the key issues of nasal administration. The toxicity of nasal mucous membrane cilia was evaluated by investigating the continuous movement ability of cilia. The main methods included an in vitro animal model method, an in vivo animal model method and an in vitro cell culture method. The mucous membrane of the Bufo gargarizans palate was similar to the mucous membrane of the human nasal cavity, and the in vitro method was simple and feasible. Therefore, in this test example, the in vitro mucous membrane of the Bufo gargarizans palate was used as a model to investigate the cilia toxicity of test drugs A-D in 1.1.

2.1 in Intro Bufo gargarizans Palate Model

First, the in vitro mucous membrane of the Bufo gargarizans palate was extracted. The spine of the Bufo gargarizans was completely destroyed and placed on a board by using a probe. The Bufo gargarizans palate was cut off. The mucous membrane of the Bufo gargarizans palate was removed carefully and isolated with ophthalmic scissors to obtain the mucous membrane of the Bufo gargarizans palate having a uniform size of about 10 mm², and carefully rinsed with normal saline. The cilia of the Bufo gargarizans palate were laid face up on a slide. The excess normal saline was sucked out. 80 μL of 4 different solutions (test drugs A-D in 1.1) were respectively dropwise added to the mucous membrane surface and gently covered with coverslips. Bubbles were drained off. 12 groups (n=12) were determined for each sample with normal saline as a negative control. The cilia morphology and swing were observed and recorded under 25×40× and 25×100× optical microscopes, respectively. Video was recorded to observe whether the mucous membrane condition was normal. Specimens were then placed in a chromatographic tank saturated with water vapor at a room temperature of 20° C. After administration, the movement state of cilia was observed and recorded under a 25×100× optical microscope at a predetermined time point.

### 2.2 Evaluation Indexes

The persistent vibration duration (PVD) from the start of administration to the complete cessation of cilia movement was recorded. The toxicity of the measured component was measured by the percentage of persistent vibration (PPV) in continuous movement of cilia, and the higher the PPV, the less cilia toxicity of the component. A formula of PPV was as follows:

$$PPV ( \% ) = {T}_{s ample} / {T}_{saline} \times 100 \%$$

Tₛₐₘₚₗₑ was the duration of cilia swing in an administration group, and Tₛₐₗᵢₙₑ was the duration of cilia swing in a normal saline group.

### 2.3 Statistical Analysis

Data results were expressed as “mean+standard deviation” (n=12). A T-test was used to test the significant difference in the effect of the PVD of cilia movement of the in vitro mucous membrane on the Bufo gargarizans palate, and p<0.05 was considered significant.

### 3. Results

TABLE 7
Effects of drugs on the cilia movement of the Bufo
gargarizans palate (n = 12, x ± s)

| Groups | PVD (h) | PPV (%) |
|---|---|---|
| A | 18.63 ± 3.75 | — |
| B | 23.67 ± 7.26 | 127.05 |
| C | 26.04 ± 6.35 | 139.77 |
| D | 23.83 ± 4.74 | 127.91 |

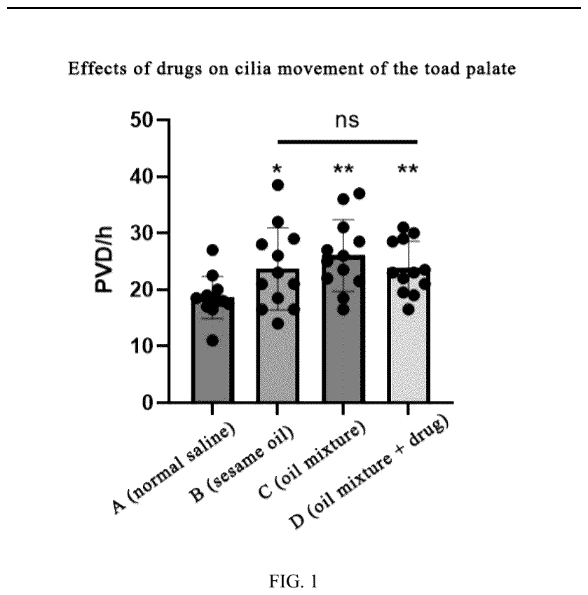

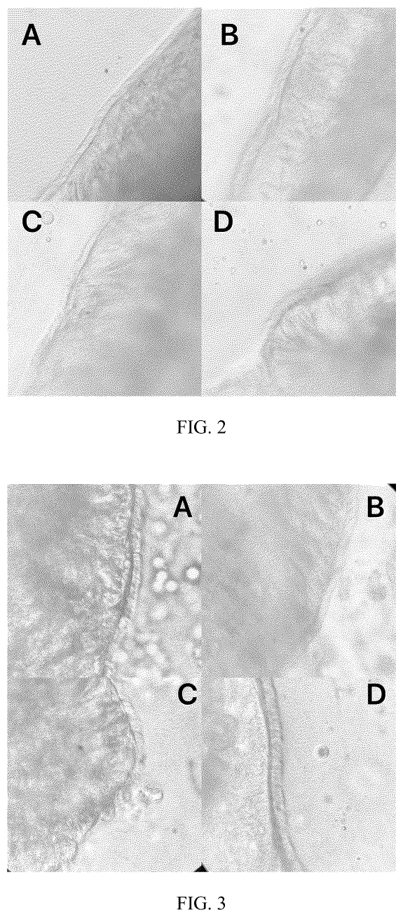

FIG. 1 and Table 7 showed the effects of different test drugs on the cilia movement of the Bufo gargarizans palate. FIG. 2 and FIG. 3 show the cilia movement states of different test drugs at the beginning of administration and after 18 h of administration, respectively.

In the present test example, the cilia toxicity of nasal mucous membrane with B (refined sesame oil), C (sardine peptide nasal spray (oil solution) in Embodiment 4) and D (sardine peptide nasal spray (oil solution)+berberine sesame oil suspension in Embodiment 4) was evaluated by using an in vitro toad palate method, and A (0.9% normal saline) was used as a negative control. The results showed that the effects of Groups B, C and D on PVD of cilia movement of the in vitro mucous membrane on the Bufo gargarizans palate were statistically significant compared with Group A (p<0.05), and there was no significant difference in T-test between Groups B and C, between Groups B and D, and between Groups C and D. Compared with Group A, the PVD of cilia movement was prolonged in Groups B, C and D, indicating that there was no acute nasal cilia toxicity in Groups B, C and D in the present test example.

The results of the study on the nasal mucous membrane cilia toxicity of the sardine peptide nasal spray (oil solution) in Embodiment 4 by an in vitro toad palate model showed that the toxicity of each prescription component of this drug on the nasal mucous membrane were lower than that of normal saline. Specifically, the PVDs of Groups B, C, and D were significantly lower than that of the negative control normal saline. Therefore, it can be preliminarily determined that the sardine peptide nasal spray (oil solution) in Embodiment 4 had good safety.

Finally, it should be noted that the above is only used to illustrate the technical solution of the invention rather than to limit it. Although the invention has been described in detail with reference to the preferred arrangement, it should be understood by those skilled in the art that the technical solution of the invention can be modified or replaced in equivalent manner without departing from the spirit and scope of the technical solution of the invention.

1. An application of a sardine peptide composition in a drug for treating allergic rhinitis, wherein the sardine peptide composition is a mixture of dipeptide to pentapeptide of sardine;
in the sardine peptide composition, the sardine dipeptide comprises Ala-Leu, Lys-Trp, Ala-Trp, Met-Leu, Arg-Tyr, Met-Phe, Gly-Trp, Met-Tyr, Gly-Tyr, Phe-Leu, Ile-Phe, Thr-Tyr, Ile-Trp, Tyr-Leu, Ile-Tyr, Val-Phe, Leu-Trp, Val-Trp, Leu-Tyr and Val-Tyr; the sardine tripeptide comprises Ala-Asn-Pro, Ala-Lys-Lys Arg-Phe-His, Arg-Val-Tyr, Gly-Arg-Pro, Ile-Val-Tyr, Trp-Ile-Thr, Tyr-Val-Pro and Val-Val-Phe; the sardine tetrapeptide is Gly-Trp-Ala-Pro; and the sardine pentapeptide is Asn-Val-Pro-Ile-Tyr.

2. The application according to claim 1, wherein the sardine peptide composition is prepared from sardine by means of a microbial fermentation method, an acid method, an alkaline method, an electrical method, an artificial grafting method, a gene expression method, and an enzymolysis method.
